(19) United States
(12) Patent Application Publication
(10) Pub. No.: US 2005/0201911 A1
(43) Pub. Date: Sep. 15, 2005

(54) Air purification system using antibody material and air purification filter using same

(75) Inventors: Hideo Namiki, Tokyo (JP); Mujo Kim, Kyoto (JP); Shinichiro Kusunoki, Tokyo (JP); Tadayoshi Taniyama, Yokohama-shi (JP)

Correspondence Address: BEYER WEAVER & THOMAS LLP, P.O. BOX 70250, OAKLAND, CA, 94612-0250, US

(73) Assignees: Waseda University; Pharma Foods International Co., Ltd.; LSL Co., Ltd.

(21) Appl. No.: 10/798,186

(22) Filed: Mar. 10, 2004

(51) Int. Cl. A61L 9/00; A62B 7/08

(52) U.S. Cl. 422/306; 422/5; 422/28; 422/120

(57) ABSTRACT

An air purification system comprises: a duct working as an air-flow path; an air-conditioning unit having an air purification filter provided on the duct; and a blower fun. The air purification filter contains an antibody material. The antibody material comprises: the blood components of a livestock animal which is administered desired antigens such as bacteria, mold, virus and environmental allergen; and/or a livestock food product containing that blood components. The blood components and the livestock food products are contained in the antibody material as effective components.

### BACKGROUND OF THE INVENTION

1. Field of the Invention

The present invention relates to an air purification system which can adsorb and remove various kinds of bacteria, mold, virus, environmental allergen or the like. The present invention also relates to an air purification filter which is suitable for the air purification system.

2. Description of the Related Art

Recently, infection from bacteria, mold, virus or the like has become a social problem. For example, infection acquired in a hospital or one that is acquired in a place where the general public gather in crowds such as a place in a public facility, etc has been apprehended. Especially, infection acquired in a hospital may cause the outbreak of MRSA (Methicillin-Resistant Staphylococcus Aureus) due to the abuse of antibiotics or the like. As a countermeasure against such infection, clothing and instruments have been conventionally disinfected.

However, it has become apparent that such disinfection alone can not effectively prevent the infection from bacteria, mold, virus or the like. To be more precise, not only in a hospital or a public facility but also in a recent building, air is circulated by an air-conditioner via ducts provided on all rooms in order to control a room temperature of the entire building, etc. Accordingly, airborne materials such as bacteria, mold, virus or the like in the building may be dispersed inside the building entirely. Conventionally, there has been no effective solution for preventing such aerial infection.

For example, one of conventional methods for sterilizing the airborne materials is providing a titanium oxide or a strongly acidic sterilization zone on an air-flow unit of the air-conditioner so as to sterilize bacteria, mold, virus or the like passing through the titanium oxide or the strongly acidic sterilization zone. This method, however, has problems such that it needs a certain amount of time for sterilization and can not achieve a sufficient effect from the standpoint of sterilization effectiveness. Moreover, sterilizing air flowing in the building entirely is not practical from the standpoint of cost. Further, for some kinds of bacteria, mold, virus or the like, the sterilization effectiveness may not be sufficiently acquired.

In addition to bacteria, mold, virus or the like, a problem due to a so-called environmental allergy which is caused by pollen or house dust such as mite has been pointed out recently. It is thus desirable to remove such allergens which cause the environmental allergy.

Therefore, it is desirable to provide an air purification system being able to remove not only various kinds of bacteria, mold, virus or the like but also the environmental allergen with a simple structure since aerial infection caused by those materials and contamination of atmosphere can be prevented.

As one which satisfies the above-explained demand, Japanese Unexamined Patent Publication No. 11-304800 discloses an allergen collecting and inspection kit. This kit collects samples of allergen presenting in air such as mite, cedar pollen into an antigen solution (to be more precise, a liquid including phosphate buffer as a primary constituent, the phosphate buffer containing interfacial active agent) and dissolves the samples into the antigen solution by allowing air containing the samples to come in contact with or pass through the antigen solution. This publication further discloses an allergen collecting and inspecting kit which comprises at least: a container; an antigen solution in the container; an air introducing means introducing air into the antigen solution, the air containing the samples; and an air exhaustion means exhausting the air contacted by the antigen solution. This kit may have a loading slot for inserting one subjected to be inspected. In this kit, a sample collecting member and an inspection member are integrated with each other, while the kit can be easily and correctly used in a general family. Especially, because a large amount of the samples can be collected directly into the antigen solution, this kit can inspect the presence of allergens sensitively. Moreover, this kit can be used with an air purifying device in order to prevent harm due to allergens. This kit, however, is one which dissolves allergens such as mite and pollen into the antigen solution, but does not deal with various other antigens than the above-described allergens, such as bacteria, mold, virus or the like. Further, since this kit uses a liquid of the antigen solution, the liquid leaks from the container when the container falls down even though it is disclosed to place a water-retentive material such as sponge, cotton, natural sponge, etc, into the container in order to prevent the antigen solution from leaking. Therefore, both a direction and a position for attaching the kit to a device such as an air purification device are limited.

Meanwhile, Japanese Unexamined Patent Publication No. 2000-14759 discloses a method for inhibiting a growth of Legionella bacteria presenting in air or water, the method allowing air or water containing Legionella bacteria to come in contact with: marine-diatom earth containing humic materials; or its extract. This method uses a fact such that marine-diatom earth containing humic materials inhibit a growth of Legionella bacteria in air or water. This method, however, can not remove bacteria, mold, virus, environmental allergen or the like in air except Legionella bacteria.

Further, Japanese Unexamined Patent Publication No. 6-91117 discloses a air purification device and a method for purifying air in a room while using: a filter containing lytic enzyme produced by myxobacteria and/or antibacterial agent; and an antibacterial polymer filter which specifically adsorbs microbes due to antibacterial and anti-mold characteristics. In this air purification device, the lytic enzyme containing filter is placed rearwardly of a dust-proof filter, while the antibacterial polymer filter is placed reawardly of the lytic enzyme containing filter. When air containing dusts and other contaminations pass through the dust-proof filter, dusts, etc, can be removed. Then the filtrated air passes through the lytic enzyme containing filter, while microbes adhered to the surface of the lytic enzyme containing filter can be sterilized by the lytic enzyme and/or antibacterial agent, and further dissolved. Resifual few bacteria or mold pass through the lytic enzyme containing filter can be collected and adsorbed by the antibacterial polymer filter. By sterilizing the residual few bacteria or mold with the antibacterial effect of the antibacterial polymer filter, bacteria, mold and bacillus or the like can be removed from air in a living space and working space. Moreover, contamination of bacillus due to an air purification device in a hospital can be prevented. The air purification device, however, has a problem such that complicated production processes for those filters are required since those filters are essential components for the air purification device. Therefore, the production of the air purification is not efficient.

One of the assignee of this application, Pharma Foods International Co., Ltd., discloses anti-inflammatory composition of matter in Japanese Unexamined Patent Publication No. 2002-213789 as a technique to utilize a livestock food product containing antibodies obtained from a livestock animal administered antigens. The anti-inflammatory composition of matter comprises a livestock food product as an effective component. The livestock food product contains blood components of a livestock animal inflamed by being administered inflammatory agents (ones which cause an inflammatory response such as pain, flare, fever, swelling, functional disorder, etc, when administered to the livestock animal) and/or changed from the blood components.

As a technique utilizing antibodies produced from the livestock food product, for example, Japanese Unexamined Patent Publication No. 8-231425 discloses a vaccine such that antibodies produced from an egg of a bird are administered to a mammal, while the bird is immunized against antigens (for example, carcinogenic, bacterial and viral seeds, living body control agents having origins thereof in a plant and an animal, toxins, poisonous materials, etc). Further, Japanese Unexamined Patent Publication No. 9-20684 discloses a chewing gum containing hen egg antibodies against: microbes (bacteria, mycoplasma, virus, etc) causing infection such as intraoral infection, throat or airway infection, and gastrointestinal infection; and allergens causing respiratory disease. Still further, Japanese Unexamined Patent Publication No. 2002-165567 discloses a method for producing a harmful-bacteria-free liquid food, the method comprising the steps of: collecting an egg from a laying hen immunized against the harmful bacteria (salmonella, Bacillus coli, yellow staphylococci, food intoxication bacteria or putrefactive bacteria such as Bacillus cereus); extracting egg yolk antibodies from an egg yolk separated from the collected egg; and allowing the egg yolk antibodies to the liquid food while holding the egg yolk antibodies into a support (for example, a bead, a non-woven cloth, a glass-wool, etc).

As explained above, attempts to utilize a livestock food product containing antibodies, hen egg antibodies or egg yolk antibodies for medical treatment of diseases due to inflammatory reaction, prevention of infection and sterilization for a liquid food has been made. However, there is no technique which utilize a livestock food product to an air purification filter, the livestock food product containing various kinds of antibodies as effective components against bacteria, mold, virus, environmental allergen or the like in air.

### SUMMARY OF THE INVENTION

The present invention has been made to solve the above problems. It is accordingly an object of the present invention to provide an air purification system which can purify air by utilizing a livestock food product containing antibodies obtained from a livestock animal administered antigens such as bacteria, mold, virus, environmental allergen or the like.

Another object thereof is to provide an air purification filter which is suitable for the air purification system.

In order to attain the above objects, according to a first aspect of the present invention, there is provided an air purification system comprising: an air-conditioning unit taking in air; an antibody material comprising a livestock food product, the livestock food product containing antibodies, the antibodies being produced by administering antigens to a livestock animal; and an adsorption member having the antibody material, wherein the adsorption member allows the antibody material to come in contact with the air taken-in by the air conditioning unit in order to adsorb and remove the antigens contained in the air.

Inventors of the present invention focused on a remarkable fact that antibodies are produced by administering antigens to a livestock animal and it is contained in a livestock food product thereof as effective components. Various kinds of the antibodies can be produced at once in accordance with the kinds of bacteria, mold, virus or environmental allergen, and thus various kinds of the antibodies can be produced at once by administering various kinds of antigens. Inventors of the present invention have found out the fact that the antibodies can adsorb and remove the antigens by allowing the livestock food product, which contains the antibodies as effective components, to come in contact with the antigens. Based on the remarkable fact, the inventors have reached the present invention.

The above-described antigens may be bacteria, mold, virus or environmental allergen.

The adsorption member may comprise a filter made from a non-woven cloth, the non-woven cloth containing the antibody material.

Alternatively, the adsorption member may comprise: a casing allowing the air taken-in by the air-conditioning unit to pass through inside thereof: and a plurality of screening plates having the antibody material, the plurality of screening plates being provided inside the casing laterally and alternately so as to come in contact with the air passing through an inside the casing.

The adsorption member may comprise a tank filled with a solution including the antibody material mixed therein.

The adsorption member may comprise: a solution containing the antibody material; and a spraying means for spraying the solution to a desired space in order to adsorb and remove the antigens contained in the air taken-in by the air-conditioning unit at the desired space.

The livestock animal may be laying hen; and the livestock food product may be egg.

The above-described livestock food product may be one of a following or a combination of two or more of the followings: liquid whole egg, liquid white egg, liquid egg yolk, dried whole egg, albumen powder, dried yolk, defatted dried whole egg or defatted dried yolk.

Alternatively, the livestock animal may be cattle; and the livestock food product may be dairy.

The above-described livestock food product may be one of the followings or a combination of two or more of the followings: defatted milk, whole milk powder or defatted dry milk.

In order to attain the above objects, according to a second aspect of the present invention, there is provided an air purification filter for purifying air comprising: an antibody material comprising a livestock food product, the livestock food product containing antibodies, the antibodies being produced by administering antigens to a livestock animal, wherein: the air purification filter purifies air by allowing air to flow therein so that the antibody material comes in contact with the air in order to adsorb and remove the antigens contained in the air; and the adsorbed and removed antigens are bacteria, mold, virus or environmental allergen.

The livestock animal may be laying hen; and the livestock food product is egg.

The above-described livestock food product may be one of the followings or a combination of two or more of the followings: liquid whole egg, liquid white egg, liquid egg yolk, dried whole egg, albumen powder, dried yolk, defatted dried whole egg or defatted dried yolk.

Alternatively, the livestock animal may be cattle; and the livestock food product may be dairy.

The above-described livestock food product may be one of the followings or a combination of two or more of the followings: defatted milk, whole milk powder or defatted dry milk.

In order to attain the above objects, according to a third aspect of the present invention, there is provided an air purification filter allowing air to flow therein comprising: an antibody material comprising a livestock food product, the livestock food product containing antibodies, the antibodies being produced by administering various kinds of antigens to a livestock animal; and a non-woven cloth containing the antibody material by being soaked into a solution of the antibody material, wherein: the air purification filter purifies air by allowing the antibody material to come in contact with the air in order to adsorb and remove the various kinds of antigens contained in the air; and the adsorbed and removed various kinds of antigens are bacteria, mold, virus or environmental allergen.

### BRIEF DESCRIPTION OF THE DRAWINGS

These objects and other objects and advantages of the present invention will become more apparent upon reading of the following detailed description and the accompanying drawings in which:

FIG. 1 is a schematic cross sectional view showing the structure of an air purification system according to a first embodiment of the present invention;

FIG. 2 is a top plan view showing the structure of an adsorption member included in the air purification system according to a second embodiment of the present invention;

FIG. 3 is a side view showing a structure of an adsorption member included in the air purification system according to a third embodiment of the present invention; and

FIG. 4 is a schematic diagram showing an experimental apparatus used in an experiment.

### DETAILED DESCRIPTION OF THE PREFERRED EMBODIMENT

A preferred embodiment of the present invention will now be described in detail with reference to the accompanying drawings.

(First Embodiment)

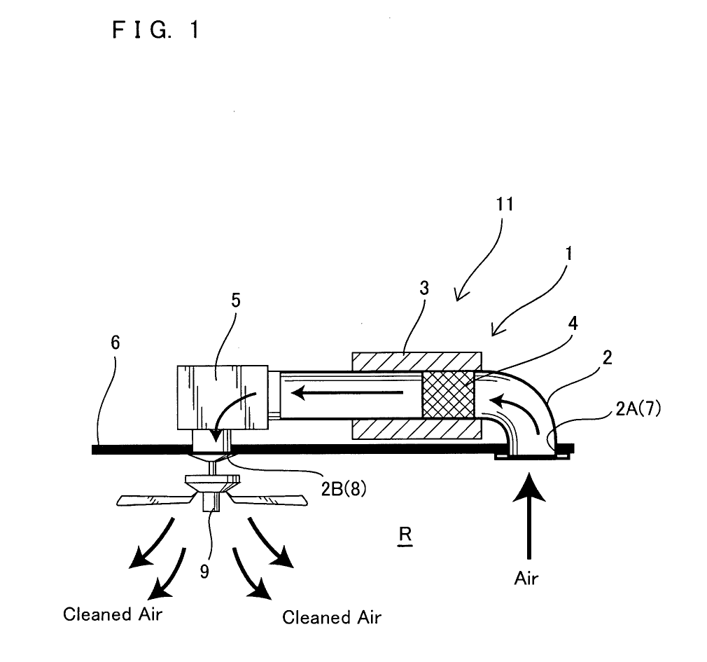

As shown in FIG. 1, according to a first embodiment of the present invention, an air purification system 1 using an antibody material comprises: a duct 2 forming an air-flow path; an air-conditioning unit 3 including an air purification filter 4 as an adsorption member 11 provided on the duct 2; and a blower fan 5. A proximal end 2A, and a distal end 2B of the duct 2 are respectively communicated with an inlet port 7 formed at a ceiling 6 as an inlet member, and an outlet port 8 working as an outlet member. A fan 9 is provided on the outlet port 8 in order to disperse air-conditioning unit 3. In this air purification system 1, the air purification filter 4 comprises a non-woven cloth containing an antibody material.

The antibody material comprises, for example, a livestock food product containing antibodies to bacteria, mold, virus, environmental allergen or the like.

Detail of the antibody material used by the air purification system 1 will now be explained. Livestock animals have capabilities for producing immune proteins (antibodies) in bloods thereof, while the antibodies specifically bind to one entering to the organisms of the livestock animal such as bacteria, mold, virus, environmental allergens and also foreign organisms such as foreign proteins so as to remove the infectiousness or the toxicities thereof (antigen-antibody reaction). Especially, in case of hen egg or dairy, the antibodies in the bloods of the livestock animal are concentrated and excreted therein. By introducing the antibodies to the human body, the antibodies can be produced therein. Meanwhile, the antibodies can be effective not only to bacteria, mold and virus but also chemical substances. The chemical substances include one acting as environmental allergen.

Inventors of the present invention had made various researches about the above-explained antibodies. As a result, they found out a fact that the antibodies remove toxicities of antigens such as bacteria, mold, virus, environmental allergen or the like when contacting those. Accordingly, the antigens can be adsorbed and removed by allowing the antibodies to exist in air so as to come in contact with the antigens.

Therefore, the air purification system 1 using the antibody material can work not only for bacteria, mold, virus but also for environmental allergen. As virus that the air purification system can adsorb and remove, influenza virus, parainfluenza virus, Respiratory Syncytial Virus (RSV), adenovirus, rhinovirus, corona virus, mumps virus, measles virus, rubella virus, smallpox virus, etc, can be considered. Moreover, coli bacteria, tuberculosis bacteria, hemophilus, diphtheria bacillus, pneumococcus, bordetella pertussis, etc, can be considered as bacteria which the air purification system 1 can adsorb and remove. Further, pollen, mite (tick) or the like can be considered as environmental allergen. Various kinds of the antibodies to those antigens can be produced at once by administering: the above-explained killed bacteria or virus, mold; or one or more kinds of environmental allergen to the livestock animal. Accordingly, by administering various kinds of antigens at once, all kinds of antibodies to the administered antigens can be produced, and thus it is not necessary to produce various kinds of antibodies individually. The produced antibodies are subjected to be included in the blood components of the livestock animal and/or the livestock food products containing the blood components.

For example, cattle, sheep, goat, laying hen (fowl), duck, quail, or the like can be considered as the livestock animal being administered the antigens. Cattle or laying hen, however, is preferable from the standpoint of productivity, while hen egg is preferable from the standpoint of mass-productivity. There is no special limitation regarding how to administer the above-explained antigens to the livestock animal. For example, the antigens can be mixed, suspended, emulsified to a solvent such as water so as to be administered by oral administration, intraperitoneal injection, intramuscular injection, intracutaneous injection, and so on. Intramuscular injection or oral administration is suitable from the standpoint of efficiency. Amount of the antigens per one administration may vary by kinds of the antigen, kinds of the livestock animal and kinds of administration method. For example, in a case administering the antigens by muscle administration, the amount of the antigens may be one such that production of the antibodies can be perceived by 1 to 3 administrations.

The blood components of the livestock animal according to the present invention are blood, blood serum, blood plasma, etc. It is preferable that those blood components are used as dry powder. Further, fractions having 100,000 to 200,000 molecular weights generated by refining the blood serum or the blood plasma while applying an appropriate method such as ultrafiltration, gel filtration and so on can be used.

The livestock food product according to the present invention is, for example, hen egg or dairy. In a case where the livestock food product is hen egg, liquid whole egg, egg while liquid, liquid egg yolk, dried whole egg, albumen powder, dried yolk, defatted dried whole egg or defatted dried yolk can be used, while defatted dried whole egg or defatted dried yolk is preferable among those. Moreover, water-soluble fractions obtained by extracting water from the defatted dried whole egg or defatted dried yolk may be used. For example, those water-soluble fractions can be obtained by: adding water to defatted dried yolk, the amount of added water being 5 to 10 times that of defatted dried yolk; agitating those; filtering those in order to obtain filtrate thereof; extracting the obtained filtrate; and spray drying/freeze drying.

Meanwhile, in a case where the livestock food product is dairy, defatted milk, whole milk powder and defatted dry milk can be used, while defatted dry milk is preferable among those. Moreover, water-soluble fractions obtained by extracting water from the defatted dry milk may be used. Further, fractions having 100,000 to 200,000 molecular weights generated by refining the water-soluble fractions while applying an appropriate method such as ultrafiltration, gel filtration and so on can be used.

Obtaining the blood from the livestock animal, hen egg or dairy can be carried out after the antibodies are produced by the above-explained method, while the production of the desirable antibodies to the antigens is identified. Once the production of the antibodies starts, the antibodies can be continuously produced for three months, and thus the antigens may be additionally administered.

As the livestock food product containing the antibodies, hen egg and processed food thereof are preferable from the standpoints of percentage content, productivity, and reproducibility of the antibodies.

The antibody material comprising the livestock food product is mixed in a material of the non-woven cloth as powder or a solution containing it, while the non-woven cloth being subjected to form the air purification filter 4. Then, the non-woven cloth can be produced by a general procedure using that material, or the air purification filter 4 can be produced by soaking the existing non-woven cloth in the solution containing the antibody material and drying it. The air purification filter 4 produced accordingly has specific adsorbability and capability of elimination which depend on the compounding ratio of the antibody material and the thickness of the air purification filter 4. Accordingly, those capabilities of the air purification filter 4 can be designed based on the desired performance of the air purification system 1. Thicker air purification filter 4 is preferable for better adsorption and elimination effects, but in a case where the thickness thereof is too thick, pressure loss becomes too large, and thus the efficiency of the air purification system 1 falls.

Next, operations of the air purification system 1 employing the above-described structure will now be explained. When the air purification system 1 starts operating, the air-conditioning unit 3 operates so that air in a room R is taken in via the inlet port 7 and flows into the air-conditioning unit 3. The air taken-in by the air-conditioning unit 3 is to pass the air purification filter 4, and thus bacteria, mold, virus, environmental allergen or the like contained in the air are adsorbed and removed at once by contacting the antibody material contained in the air purification filter 4, while the air-conditioning unit 3 controls temperature of the air after the antigens are removed. The blower fan 5 blows the purified air so that it flows back to the room R. The fan 9 allows the purified air to spread in the room R entirely. By repeating those operations, air in the room R can be purified. The duration of the adsorption-and-elimination effect of the antibody material varies in changes with a degree of contaminations of bacteria, mold, virus, environmental allergen or the like. Accordingly, the duration of the antibody material may be previously calculated based on the fact how much the room R is dirty. On calculation, when the durability of the antibody material lasts, for example, for four months, the air purification filter 4 may be replaced in shorter period than the duration thereof, for example, every three months.

Meanwhile, a case where the air purification system 1 is applied for internally circulating air within a building is explained in this embodiment. It is, however, obvious that the air purification system 1 can be applied for introducing air from outside of a building so as to circulate it inside the building.

(Second embodiment)

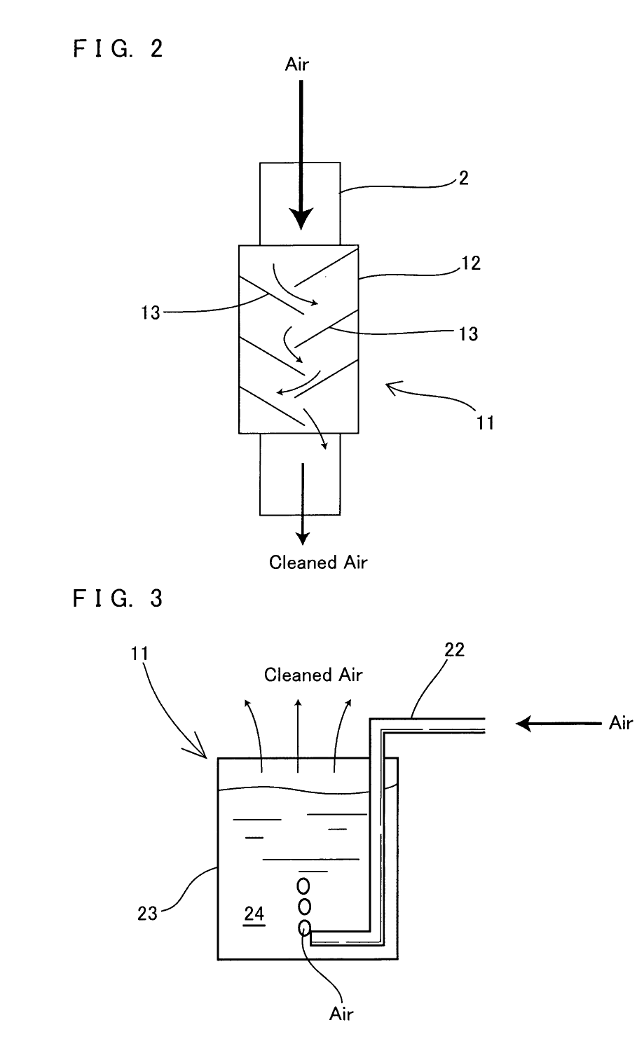

Next, a second embodiment of the present invention will now be explained with reference to FIG. 2. The air purification system 1 according to this embodiment employs essentially the same structure as that of the first embodiment except the air purification filter 4, and thus detailed explanation of the structure will be omitted.

The adsorption member 11 in the first embodiment comprises the air purification 4. On the other hand, according to the second embodiment, the adsorption member 11 may comprise a casing 12 sequentially connected to the duct 2 and a plurality of screening plates 13 provided inside the casing 12 longitudinally and alternately. The plurality of screening plates 13 has the antibody material. By employing this structure on the adsorption member 11, air in the room R is taken in via the inlet port 7 by the activated air-conditioning unit 3. The air taken-in by the air-conditioning unit 3 is to pass the adsorption member 11 while coming in contact with the plurality of the screening plates 13. Consequently, bacteria, mold, virus, environmental allergen or the like contained in the air can be adsorbed and removed at once by contacting the antibody material of the plurality of the screening plates 13, while the air-conditioning unit 3 controls temperature of the air after the antigens are removed. The blower fan 5 blows the purified air so that it flows back to the room R. The fan 9 allows the purified air to spread in the room R entirely. By repeating those operations, air in the room R can be purified.

(Third Embodiment)

Explanation will now be made to the air purification system of the present invention according to a third embodiment with reference to FIG. 3. As well as the second embodiment, the air purification system 1 according to this embodiment employs the same structure as that of the first embodiment except the air purification filter 4, and thus detailed explanation of the structure will be omitted.

In this embodiment, the adsorption member 11 comprises flow tube 22 sequentially connected to the duct 2 (not illustrated) and an adsorption-and-elimination tank 23 connected to one end of the flow tube 22. The adsorption-and-elimination tank 23 is filled with an adsorption-and-elimination solution 24 containing the antibody material. By employing this structure on the adsorption member 11, air in the room R is inhaled via the inlet port 7 by the activated air-conditioning unit 3. The air taken-in by the air-conditioning unit 3 is to be exhausted from one end of the flow tube 22 and pass the adsorption-and-elimination solution 24, and thus bacteria, mold, virus, environmental allergen or the like contained in the air can be adsorbed and removed at once by contacting the antibody material contained in the adsorption-and-elimination solution 24, while the air-conditioning unit 3 controls temperature of the air after the antigens are adsorbed and removed. The blower fan 5 blows the purified air so that it flows back to the room R. The diffusion fan 9 allows the purified air to spread in the room R entirely. By repeating those operations, air in the room R can be purified.

The present invention is not limited to the above embodiments. For example, whilst the air purification system is configured so as to be fixed to a building in the first embodiment, it may be configured as portable type. Moreover, the air purification system of the present invention may employ a structure such that a solution of the antibody material is filled in a spray in order to purify a desired space by spraying the solution thereto.

Various embodiments and changes may be made thereonto without departing from the broad spirit and scope of the invention. The above-described embodiments are intended to illustrate the present invention, not to limit the scope of the present invention. The scope of the present invention is shown by the attached claims rather than the embodiments. Various modifications made within the meaning of an equivalent of the claims of the invention within the claims are to be regarded to be in the scope of the present invention.

### EXAMPLE

The present invention will now be explained in more detail with the following example.

(Preparation of Antibody Materials)

1ml of killed coli bacteria 10⁸ cells/ml) is administered to a white leghorn laying hen (180 days past since hatched) by intramuscular injection. 8 weeks later from the first injection, a second intramuscular injection is carried out in accordance with the same method as that of the first injection. Then, all eggs that the laying hen blows at 10 weeks later from the first injection are opened in order to obtain egg yolks. The egg yolks are separated and homogenized, while dry powder of the egg yolks is obtained by spray drying. The egg yolk dry powder are defatted by ethanol so as to delete fat thereof, and then dried out under reduced pressure. Defatted dried yolk as the antibody material can be obtained.

(Manufacture of the Air Purification Filter)

100 g of the obtained defatted dried yolk is suspended into 900 ml of water and agitated for 30 minutes. The solution is then filtrated in order to obtain filtrate thereof. The filtrate is concentrated. A non-woven cloth is soaked into the concentrated filtrate and then dried. The dried non-woven cloth is stuck to a frame having a predetermined shape, thereby manufacturing the air purification filter.

(Air Purifying Experiment)

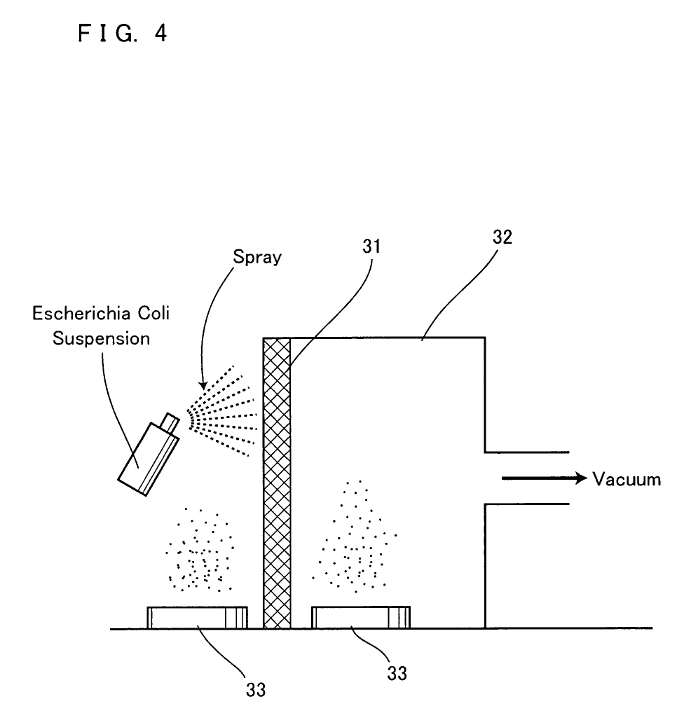

Coli bacteria (wild type) are suspended to water so that coli bacteria suspension (10⁸ cells/ml) is processed. The coli bacteria suspension is poured in a spray. As shown in FIG. 4, the coli bacteria suspension is sprayed to one surface of a box 32 for 10 seconds from the outside the box 32, the box 32 employing a structure such that one surface thereof being the air purification filter 31. The box 32 also employs a structure such that it is able to take in air toward the other surface thereof.

At this time, Petri dishes 33 filled with water are provided front and rear of the air purification filter 31 for 30 seconds. Water in the Petri dishes 33 is diluted properly, and then cultured at LB culture medium. Next, colonies of coli bacteria are counted. Result of count is shown in graph 1 (example 1). Likewise, regarding a case where the air purification filter 31 is made from the non-woven cloth which is the same as that of the experiment 1 but not soaked into the solution of the antibody material, Petri dishes 33 filled with water are provided front and rear of the air purification filter 31 for 30 seconds. Water in the Petri dishes 33 is diluted properly, and then cultured at LB culture medium. Next, colonies of coli bacteria are counted. Result of count is shown in graph 1 (comparative example 1).

Graph 1

Positions relative to
the air purification filter

| | Front | Rear |
|---|---|---|
| Example 1 | 3 × 10⁶ cells | 5 × 10³ cells |
| Comparative example 1 | 3 × 10⁶ cells | 1.5 × 10⁶ cells |

It is obvious from the result of the air purifying experiment that the air purification system of the present invention has superior adsorption-and-elimination effect.

1. An air purification system comprising:
an air-conditioning unit taking in air;
an antibody material comprising a livestock food product, said livestock food product containing antibodies, said antibodies being produced by administering antigens to a livestock animal; and
an adsorption member having said antibody material,
wherein said adsorption member allows said antibody material to come in contact with the air taken-in by said air conditioning unit in order to adsorb and remove said antigens contained in the air.

2. The air purification system according to claim 1, wherein said antigens are bacteria, mold, virus and environmental allergen.

3. The air purification system according to claim 2, wherein said adsorption member comprises a filter made from a non-woven cloth, said non-woven cloth containing said antibody material.

4. The air purification system according to claim 2, wherein said adsorption member comprises:
a casing allowing the air taken-in by said air-conditioning unit to pass through an inside thereof: and
a plurality of screening plates having said antibody material, said plurality of screening plates being provided inside said casing laterally and alternately so as to come in contact with the air passing through inside said casing.

5. The air purification system according to claim 2, wherein said adsorption member comprises a tank filled with a solution including said antibody material mixed therein.

6. The air purification system according to claim 2, wherein said adsorption member comprises:
a solution containing said antibody material; and
a spraying means for spraying said solution to a desired space in order to adsorb and remove said antigens contained in the air taken-in by said air-conditioning unit at the desired space.

7. The air purification system according to claim 2, wherein:
said livestock animal is laying hen; and
said livestock food product is hen egg.

8. The air purification system according to claim 7, wherein said livestock food product is one of the followings or a combination of two or more of the followings: liquid whole egg, liquid white egg, liquid egg yolk, dried whole egg, albumen powder, dried yolk, defatted dried whole egg or defatted dried yolk.

9. The air purification system according to claim 2, wherein:
said livestock animal is cattle; and
said livestock food product is dairy.

10. The air purification system according to claim 9, wherein said livestock food product is one of the followings or a combination of two or more of the followings: defatted milk, whole milk powder or defatted dry milk.

11. An air purification filter for purifying air comprising:
an antibody material comprising a livestock food product, said livestock food product containing antibodies, said antibodies being produced by administering antigens to a livestock animal, wherein:
said air purification filter purifies air by allowing air to flow therein so that said antibody material comes in contact with the air in order to adsorb and remove said antigens contained in the air; and
said adsorbed and removed antigens are bacteria, mold, virus or environmental allergen.

12. The air purification filter using the antibody material according to claim 11, wherein:
said livestock animal is laying hen; and
said livestock food product is hen egg.

13. The air purification filter using the antibody material according to claim 12, wherein said livestock food product is one of the followings or a combination of two or more of the followings: liquid whole egg, liquid white egg, liquid egg yolk, dried whole egg, albumen powder, dried yolk, defatted dried whole egg or defatted dried yolk.

14. The air purification filter using the antibody material according to claim 11, wherein:
said livestock animal is cattle; and
said livestock food product is dairy.

15. The air purification filter using the antibody material according to claim 14, wherein said livestock food product is one of the followings or a combination of two or more of the followings: defatted milk, whole milk powder or defatted dry milk.

16. An air purification filter allowing air to flow therein comprising:
an antibody material comprising a livestock food product, said livestock food product containing antibodies, said antibodies being produced by administering various kinds of antigens to a livestock animal; and
a non-woven cloth containing said antibody material by being soaked into a solution of said antibody material, wherein:
said air purification filter purifies air by allowing said antibody material to come in contact with the air in order to adsorb and remove said various kinds of antigens contained in the air; and
said adsorbed and removed various kinds of antigens are bacteria, mold, virus or environmental allergen.
